(19) United States
(12) Patent Application Publication
(10) Pub. No.: US 2020/0245861 A1
(43) Pub. Date: Aug. 6, 2020

(54) System and Method for Determining Characteristic Values of Ametropia of a Test Person

(71) Applicant: TECHNISCHE HOCHSCHULE KÖLN, Koln (DE)

(72) Inventors: Uwe Oberheide, Köln (DE); Gero Wiel, Leverkusen (DE)

(21) Appl. No.: 16/754,038

(22) PCT Filed: Oct. 5, 2018
(86) PCT No.: PCT/EP2018/077219

§ 371 Date: Apr. 6, 2020

(30) Foreign Application Priority Data
Oct. 6, 2017 (DE) 10 2017 123 301.6

(51) Int. Cl. A61B 3/032; A61B 3/00; G02C 7/02

(52) U.S. Cl. CPC A61B 3/032; G02C 7/02; A61B 3/0008

(57) ABSTRACT

The disclosure relates to a system for determining characteristic values of ametropia of a test person, comprising a device for generating optical test structures and a device for observing the test structures by the test person, said device comprising a correction unit for correcting possible ametropia phenomena of the test person. According to the disclosure, the optical test structures have at least two moving speckle patterns of different wavelengths. The disclosure further relates to a corresponding device for generating optical test structures for determining characteristic values of ametropia of a test person, and to a corresponding method for determining characteristic values of ametropia of a test person.

### INTRODUCTION

The disclosure relates to a system for determining characteristic values of ametropia of a test person, comprising (i) a device for generating optical test structures and (ii) a device for observing the test structures by the test person which device comprises a correction unit for correcting possible ametropia phenomena of the test person.

The disclosure further relates to a corresponding device for generating optical test structures for determining characteristic values of an ametropia of a test person and to a corresponding method for determining characteristic values of an ametropia of a test person.

Such a system is known as a phoropter system. The phoropter is an ophthalmic optical device by means of which the so-called subjective refraction of a test person can be determined. This is usually determined based on objectively measured vision defects (ametropia) and is required for fitting eyeglass lenses or contact lenses. To this end, the test person looks from the back of the device through two round openings, the distance of which can be adjusted according to the individual eye distance. The examiner makes the necessary adjustments from the front.

In medical optics, ametropia refers to the state of an eyeball that does not sharply image an object disposed optically in the infinite with relaxed accommodation onto the retina. A distinction is made between the following cases: (a) hyperopia (also called farsightedness) refers to an ametropia in which the image of an object disposed optically in the infinite comes to lie behind the retina with relaxed accommodation when using parallel incident light beams. (b) Myopia (also called shortsightedness) refers to an ametropia in which the image of the object disposed optically in the infinite comes to lie in front of the retina with relaxed accommodation. Hyperopia and myopia are denoted together as “axis ametropias”. (c) Astigmatism (also known as corneal irregularity) refers to the condition of an eyeball in which parallel incident light beams are refracted differently depending on their plane of incidence. The planes with maximum and minimum refractive power are usually perpendicular to each other. The difference in refractive power between these two planes is denoted as the strength of astigmatism.

The determination of characteristic values of ametropia of a test person is nothing more than the determination of the subjective refraction (often also called “adjustment” or “adjustment of eyeglasses”) of the test person. Different lenses are consecutively and systematically provided to the test person and he is asked for an improvement or worsening of the visual impression. As a rule, those optotypes that are also used for determining the visual acuity are offered as test structures for viewing by means of a projection device.

The selection and provision of the lenses can be accelerated by use of a phoropter. The use of a phoropter offers a convenient way to offer adults and older children different eyeglass lens thicknesses in increments of 0.25 diopters and thus to check whether this changes the visual acuity. Here, all spherical and cylindrical glass thicknesses required for determining the eyeglass lenses are available. The setting of the axial position of cylindrical glasses corresponds to the so-called TABO scheme from 0° to 180°. The phoropter is operated either manually by use of rotary knobs attached to the front face of the device or computer-controlled by use of a central control panel connected to the device. In this case, it is also possible via an interface to transmit values stored in the computer, for example previous eyeglass values or the results of an objective refraction measurement, directly to the phoropter, which then adjusts these values.

Moreover, it is possible with a phoropter to implement examinations of double-vision with regard to heterophoria, simultaneous viewing and fusion by use of color and polarization filters. Built-in prisms are available for correction, as well as sieve tests for special orthoptic examinations (Worth test, Schober test).

### SUMMARY

It is an object of the disclosure, according to an embodiment, to provide measures by means of which a determination of characteristic values of an ametropia is also possible for smaller children and/or persons with reduced visual capacity.

In the system for determining characteristic values of an ametropia of a test person, which comprises a device for generating optical test structures and a device for observing the test structures by the test person, which in turn includes a correction unit for correcting possible ametropia phenomena of the test person, it is provided according to an embodiment of the disclosure that the optical test structures have at least two moving speckle patterns of different wavelengths. In this context, moving means moving relative to the head or eyes of the test person. The system is a system for determining characteristic values of the ametropia of the test person by means of subjective refraction.

In the disclosure, the determination of the ametropia is used by manually determining the direction of movement of “speckle” phenomena (virtual light structures). In the case of a relative motion head-to-speckle pattern for a shortsighted person the perceived pattern moves in the opposite direction, for a farsighted person in the same direction as the pattern itself.

In the disclosure, different, simultaneously shown speckle patterns of different color or wavelength are used as an extension. Here, the chromatic aberration of lenses is used, which ensures that light of different wavelengths or color is refracted differently. From the directions of movement, by comparing two different wavelengths, ideally red and green, each of which creates a pattern, the best subjectively perceived correction can be determined. With optimal correction, the two speckle patterns do not move in the same direction, but in different directions. By this (based on mathematical simulations of the light paths) an accuracy of 0.25 dpt—corresponding to the accuracy of test eyeglasses or phoropters—can be achieved.

In the system it is in particular provided that the device for generating optical test structures, per an embodiment, is configured as a projection device for projecting coherent light with at least two different wavelengths onto a surface forming the test structures. The wavelengths correspond in particular to the colors red (approximately 650 nm) and green (approximately 500 nm).

The device for generating optical test structures is, per an embodiment, in particular adapted to generate the movement of the speckle patterns through a relative movement of the surface with respect to light beams of light with the different wavelengths incident onto the surface.

According to an embodiment of the disclosure, the correction unit comprises at least one set of different correction lenses for correcting possible ametropia phenomena of the test person. The means for observing the test structures by the test person are, in particular, so-called test eyeglasses or a phoropter.

In the device according to the disclosure for generating optical test structures for determining characteristic values of an ametropia of a test person it is provided, per an embodiment, that the test structures comprise at least two moving speckle patterns of different wavelengths. In other words, the device is a device for generating optical test structures comprising at least two moving speckle patterns of different wavelengths. The device is preferably, per an embodiment, a device for an aforementioned system for determining characteristic values of an ametropia of a test person. The determination of characteristic values of the ametropia of a test person is a determination by means of subjective refraction.

According to an embodiment of the disclosure it is provided that the device is configured as a projection device for projecting coherent light with at least two different wavelengths onto an (image or projection) surface forming the test structure.

According to a further embodiment of the disclosure it is provided that the device is adapted to generate the movement of the speckle pattern by means of a relative movement of the surface with respect to light beams of light of the different wavelengths incident on the surface. To this end, the surface comprises a sufficient surface roughness or another structure.

Here, it is provided, per an embodiment, that the device comprises at least one deflection unit moving the light beams of light of different wavelengths. This deflection unit is preferably configured in the form of a polygon scanner or galvo scanner.

According to an embodiment of the device according to the disclosure it is provided that the device comprises an element which provides the surface, in particular an element comprising a holographic structure.

According to a further embodiment of the device according to the disclosure, the device comprises an apparatus for moving the element which provides the surface.

In the method according to an embodiment of the disclosure for determining characteristic values of an ametropia of a test person the following steps are provided: (i) generating optical test structures, wherein the optical test structures comprise at least two moving speckle patterns of different wavelengths, and (ii) observing the test structures by the test person, wherein a correction of the ametropia phenomena of the test person is carried out by use of a correction unit for correcting possible ametropia phenomena. The method is a method for determining characteristic values of the ametropia of the test person by means of subjective refraction.

According to an embodiment of the disclosure it is provided that the method is carried out by means of the abovementioned system. In other words, this means the use of the abovementioned system for determining characteristic values of an ametropia (of the eyes) of a test person.

### BRIEF DESCRIPTION OF THE FIGURES

In the following, the disclosure will be explained by way of example with reference to the attached drawings based on exemplary embodiments, wherein the features shown below, both individually and in combination, may represent an aspect of the disclosure. In the drawing:

FIG. 1 shows a schematic representation of a farsighted eye observing speckle patterns;

FIG. 2 shows a schematic representation of an emmentrope eye observing speckle patterns;

FIG. 3 shows a system for determining characteristic values of an ametropia of a test person according to an embodiment of the disclosure;

FIG. 4 shows a first embodiment of a deflection unit of a device for generating moving speckle patterns;

FIG. 5 shows a second embodiment of a deflection unit of a device for generating moving speckle patterns;

FIG. 6 shows an embodiment of an apparatus for moving the screen which forms the speckle patterns; and

FIG. 7 shows a further device for generating moving speckle patterns.

### DETAILED DESCRIPTION

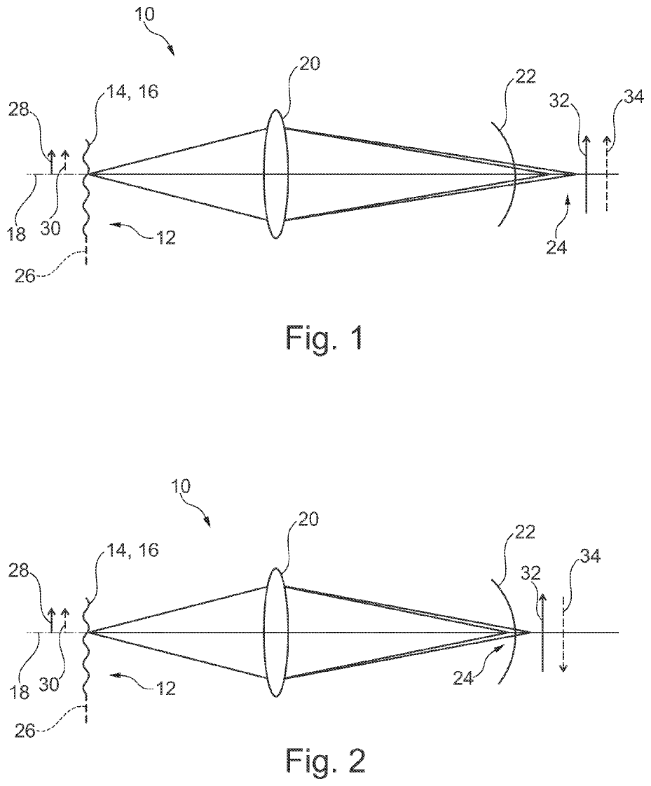

FIG. 1 shows a schematic representation of a farsighted eye 10 of a test person who observes optical test structures 12 comprising two speckle patterns 14, 16 each moving with respect to the eye 10 or the head of the test person. The eye 10 is defined via its optical axis 18, its lens 20 and its retina 22. The cornea of the eye 10 and other details are omitted from this greatly simplified illustration. Since it is a farsighted eye 10, its focus 24 lies behind the retina 22. The two speckle patterns 14, 16 move in the same object plane 26 but have different colors or wavelengths. The movement of the speckle pattern 14 of higher wavelength (for example 650 nm—red) is indicated by the arrow 28, the movement of the speckle pattern 16 of lower wavelength (for example 500 nm—green) is indicated by the arrow 30. In the example shown, the speckle patterns 14, 16 move at the same speed in the same direction. The movement of the speckle patterns 14, 16 perceived by the test person is indicated by the arrows 32, 34. Here, arrow 32 indicates the perceived movement of the speckle pattern 14 of higher wavelength and arrow 34 indicates the perceived movement of the speckle pattern 16 of lower wavelength. The farsighted test person clearly perceives the movement of the speckle patterns 14, 16, wherein the perceived direction of movement corresponds to the actual direction of movement of the speckle patterns 14, 16. This is because the focus 24 is located behind the retina 22.

The situation is different for the shortsighted eye 10 (not shown). Here the focus 24 lies in front of the retina 22. The shortsighted test person also clearly perceives the movement of the speckle patterns 14, 16, wherein, however, the perceived direction of movement compared to the actual direction of movement of the speckle patterns 14, 16 is reversed.

FIG. 2 now shows the situation corresponding to FIG. 1 for the emmentropic eye 10 and for the farsighted eye 10, in which the hyperopia is completely compensated by an additional lens, for example a contact lens or eyeglasses. In the absence of ametropia or in the case of a complete compensation of the ametropia, now the chromatic aberration of the optical system (for example the lens 20) comes into play. If the ametropia for a medium wavelength (between the higher and the lower wavelength) is completely corrected, the perceived direction of movement of the speckle pattern 14 of higher wavelength continues to correspond to the actual direction of movement of this speckle pattern 14, whereas the perceived direction of movement of the speckle pattern 16 of lower wavelength is reversed with respect to the actual direction of movement of this speckle pattern 16. In this situation, the test person has the impression that both speckle patterns 14, 16 move in opposite directions, or that both speckle patterns 14, 16 are fixed.

This phenomenon can now be used by use of a system 36 described below for determining characteristic values of the ametropia of the test person.

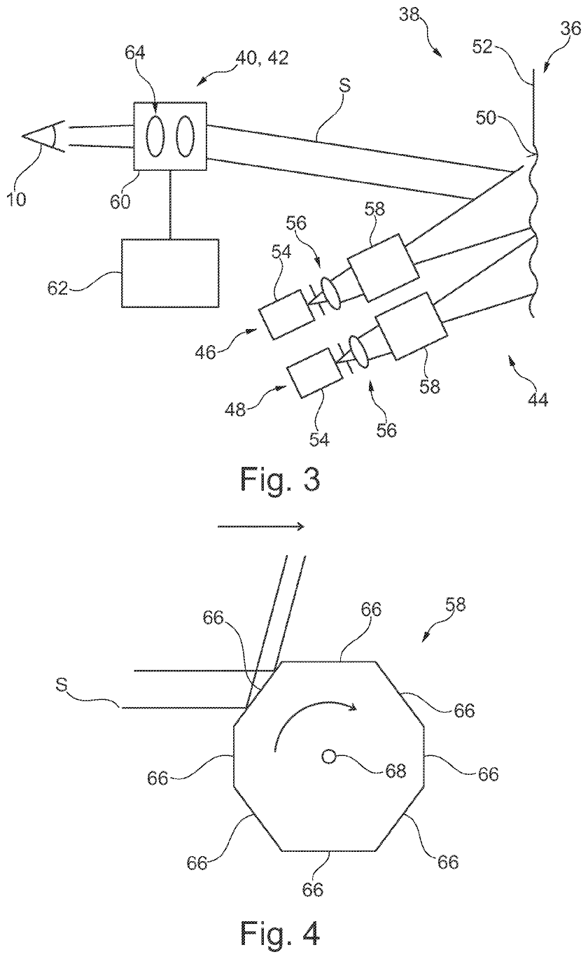

FIG. 3 shows an example of such a system 36 for determining characteristic values of an ametropia of a test person. The system comprises (i) a device 38 for generating the optical test structures 12 with the two moving speckle patterns 14, 16 of different wavelengths and (ii) a device 42 configured as a phoropter 40 for observing these test structures 12 by the test person.

The device 38 for generating the optical test structures 12 with the two moving speckle patterns 14, 16 of different wavelengths comprises a projection device 44 with two projection modules 46, 48 for projecting coherent light with different wavelengths and a screen surface 50 forming the test structures. This surface 50 is formed, for example, by a screen (also called projection screen) 52 with a corresponding surface structure. The screen surface 50 or screen 52 can be considered as a separate system component of the system 36 or as a part of the device 38. Each of the projection modules 46, 48 comprises a light source 54 for coherent light of a respective wavelength, a respective optics 56 downstream of the light source 54 and a respective deflection unit 58 for moving the light beams of light of a corresponding wavelength. Thus, an optical path S is obtained.

The device 42 for observing test structures comprises a correction unit 60 for correcting possible ametropia phenomena of the test person and an actuating element 62 for actuating the correction unit 60. The correction unit 60 comprises a set of different correction lenses 64 for the correction.

In order to determine characteristic values of the ametropia of the eyes 10 of a test person, (i) the device 38 generates optical test structures 12, wherein the optical test structures 12 comprise two moving speckle patterns 14, 16 of different wavelengths, (ii) these test structures 12 are observed by the test person via the observation device 42 which comprises the correction unit 60 and (iii) corrections of the ametropia phenomena of the test person are carried out by means of the correction unit 60.

FIG. 4 shows a first embodiment of the deflection unit 58 of the device 38 for generating moving speckle patterns 14, 16. In this embodiment, the deflection unit 58 is configured as a mirror system in the manner of a polygon scanner. In the example shown, eight mirror elements 66 are arranged in the form of an octagon, which is rotatably mounted or rotated about the axis of rotation 68 (arrow). In this way one or more light beams can be moved.

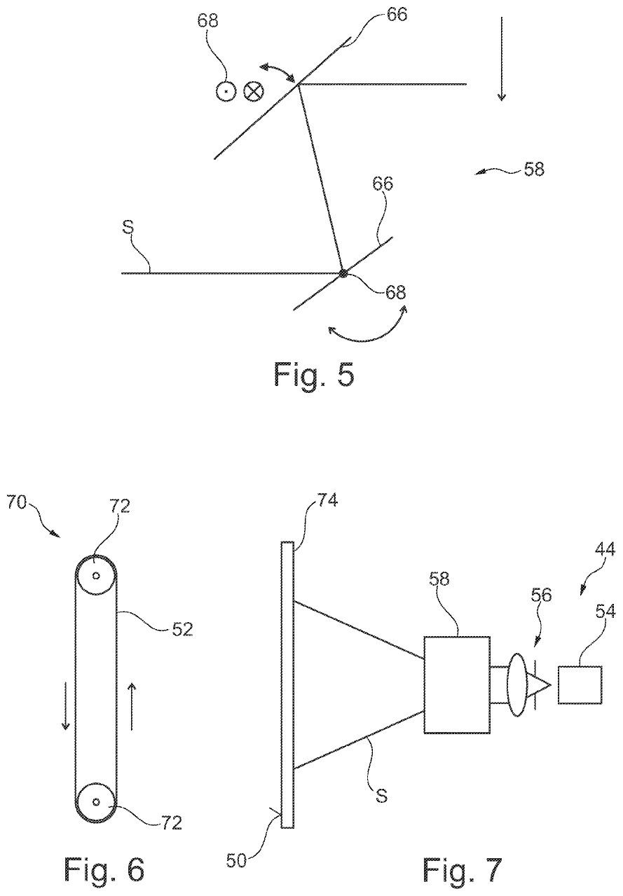

FIG. 5 shows a second embodiment of the deflection unit 58 of the device for generating moving speckle patterns 14, 16. In this embodiment, the deflection unit 58 is configured as a mirror system in the manner of a galvo scanner. In the example shown, two mirror elements 66 (x and y mirror element) are rotatably mounted about corresponding axes of rotation 68. In this way one or more light beams can be moved.

FIG. 6 shows an embodiment of an apparatus 70 for moving the screen 52 which forms the speckle patterns 14, 16. This is formed as a screen in the form of a circumferential means wrapped around two rolls 72. At least one of the rolls 72 is configured as a drive roll and causes the screen to circulate around the rolls 72 (arrow).

FIG. 7 finally shows a further device 38 for generating moving speckle pattern 14, 16. This comprises a projection device 44 with two light sources 54 for coherent light of corresponding wavelengths, optics 56 downstream of the light sources 54 and a deflection unit 58 for moving the light beams of the light as well as an element 74 providing the screen surface 50, which comprises a holographic structure, i.e. a hologram. The use of an element 74 which comprises a holographic structure for providing the screen surface 50 enables—as shown here—an illumination from the rear side, i.e. from the side of the element 74 facing away from the face 50 by the projection device 44.

It is to be understood that the foregoing is a description of one or more preferred exemplary embodiments of the invention. The invention is not limited to the particular embodiment(s) disclosed herein, but rather is defined solely by the claims. Furthermore, the statements contained in the foregoing description relate to particular embodiments and are not to be construed as limitations on the scope of the invention or on the definition of terms used in the claims, except where a term or phrase is expressly defined above. Various other embodiments and various changes and modifications to the disclosed embodiment(s) will become apparent to those skilled in the art. All such other embodiments, changes, and modifications are intended to come within the scope of the appended claims.

As used in this specification and claims, the terms “for example,” “for instance,” “such as,” and “like,” and the verbs “comprising,” “having,” “including,” and their other verb forms, when used in conjunction with a listing of one or more components or other items, are each to be construed as open-ended, meaning that the listing is not to be considered as excluding other, additional components or items. Other terms are to be construed using their broadest reasonable meaning unless they are used in a context that requires a different interpretation.

### REFERENCE SYMBOLS

10 eye
12 optical test structure
14 speckle pattern
16 speckle pattern
18 optical axis (eye)
20 lens (eye)
22 retina
24 focus
26 object plane
28 arrow (movement of speckle pattern of higher wavelength)
30 arrow (movement of speckle pattern of lower wavelength)
32 arrow (perceived movement of speckle pattern of higher wavelength)
34 arrow (perceived movement of speckle pattern of lower wavelength)
36 system for determining characteristic values of an ametropia
38 device for generating optical test structures
40 phoropter
42 device for observing test structures
44 projection device
46 projection module
48 projection module
50 screen surface
52 screen
54 light source
56 optics
58 deflection unit
60 correction unit
62 actuating element
64 set of correction lenses
66 mirror
68 axis of rotation
70 apparatus for moving the screen
72 rolls
74 hologram
S optical path

1. System for determining characteristic values of an ametropia of a test person, comprising:
a device for generating optical test structures; and
a device for observing the test structures by the test person, wherein the device comprises a correction unit for correcting possible ametropia phenomena of the test person,
wherein the optical test structures comprise at least two simultaneously shown and moving speckle patterns of different wavelengths.

2. System according to claim 1, wherein the device for generating optical test structures is configured as a projection device for projecting coherent light with at least two different wavelengths onto a screen surface which forms the test structures.

3. System according to claim 1, wherein the correction unit comprises at least one set of different correction lenses for correcting possible ametropia phenomena of the test person.

4. Device for generating optical test structures for determining characteristic values of an ametropia of a test person, according to claim 1, wherein the test structures comprise at least two simultaneously shown and moving speckle patterns with different wavelengths.

5. Device according to claim 4, wherein the device is configured as a projection device for projecting coherent light with at least two different wavelengths onto a screen surface which forms the test structures.

6. Device according to claim 5, wherein the device is adapted to generate the movement of the speckle pattern by a relative movement of the screen surface with respect to light beams of light of different wavelengths incident on this surface.

7. Device according to claim 6, wherein the device comprises at least one deflection unit for moving the light beams of light of the different wavelengths.

8. Device according to claim 5, wherein the device comprises an element providing the screen surface.

9. Device according to claim 8, wherein the device comprises an apparatus for moving the element providing the screen surface.

10. Method for determining characteristic values of an ametropia of a test person, the method comprising:
generating optical test structures, wherein the optical test structures comprise at least two simultaneously shown and moving speckle patterns of different wavelengths;
observing the test structures by the test person; and
correcting the ametropia phenomena of the test person by means of a correction unit for correcting possible ametropia phenomena.

11. Device according to claim 4, wherein the device is a device for a system according to claim 1.

12. Device according to claim 8, wherein the element comprises a holographic structure.
